(19) United States
(12) Patent Application Publication
(10) Pub. No.: US 2019/0224260 A1
(43) Pub. Date: Jul. 25, 2019

(54) Metabolic Syndrome Inhibitor

(71) Applicant: J-OIL MILLS, INC., Tokyo (JP)

(72) Inventors: Shoji MATSUMOTO, Tokyo (JP); Saki NISHIMURA, Tokyo (JP); Masayoshi SAKAINO, Tokyo (JP); Takatoshi YAMASHITA, Tokyo (JP)

(21) Appl. No.: 16/314,226

(22) PCT Filed: Jun. 26, 2017
(86) PCT No.: PCT/JP2017/023332

§ 371 Date: Dec. 28, 2018

(30) Foreign Application Priority Data
Jul. 13, 2016 (JP) 2016-138507

(51) Int. Cl. A61K 36/48; A23L 33/115; A23L 33/11; A23D 9/00; A61P 3/04

(52) U.S. Cl. CPC A61K 36/48; A23L 33/115; A23V 2002/00; A23D 9/00; A61P 3/04; A23L 33/11

(57) ABSTRACT

[Problem] To provide a metabolic syndrome inhibitor which can inhibit the accumulation of visceral fat and body fat to thereby ameliorate or prevent metabolic syndrome.

[Solution] The metabolic syndrome inhibitor according to the present invention comprises soybean hypocotyl oil as an active ingredient. In particular, the metabolic syndrome inhibitor serves as a body fat accumulation inhibitor and/or a blood neutral fat increase inhibitor. In particular, the body fat accumulation inhibitor serves as a visceral fat accumulation inhibitor.

### TECHNICAL FIELD

The present invention relates to a metabolic syndrome inhibitor, more particularly a metabolic syndrome inhibitor comprising a soybean hypocotyl oil as an active ingredient.

### BACKGROUND ART

The number of so-called obese persons whose body fat accumulates more than necessary is increasing by synergy of increased lipid energy intake rate due to change of foods and excessive intake of foods and decreased consumption of intake enemy due to life inclined to be hypokinetic. Body fats include subcutaneous fats which accumulate under a skin, and visceral fats which accumulate around an internal organ in an intestine and an abdominal cavity. When the visceral fat excessively accumulates and lifestyle diseases such as hypertension, diabetes and dyslipidemia are compositely developed, the symptom is diagnosed as metabolic syndrome.

The current diagnostic criteria for metabolic syndrome is that the followings are fulfilled:

(i) visceral fat type obesity with a waist circumference exceeding a reference value (male: 85 cm, female: 90 cm); and

(ii) at least two of the following abnormalities:
(1) a neutral fat value of 150 mg/dL or higher, and/or an HDL-cholesterol value of lower than 40 mg/dL,
(2) a systolic blood pressure of 130 mmHg or higher, and/or a diastolic blood pressure of 85 mmHg or higher, and
(3) a fasting blood glucose of 110 mg/dL or higher.

A person with metabolic syndrome is likely to advance arteriosclerosis, and as a result, the person is susceptible to serious diseases such as myocardial infarction and cerebral infarction. It is important to prevent and solve metabolic syndrome from the viewpoint of preventing these diseases.

For preventing metabolic syndrome, attention is generally directed to a solution to inactivity, and improvement of dietary habit for lowering a lipid energy intake rate, e.g. diet, intake of low-fat foods, and cooking method using fats as little as possible.

It has been also proposed to reduce body fats not by improvement of the lifestyle but by oral intake of a sate body fat-reducing agent. For example, body fat-reducing agents and body fat-burning agents comprising a dioxabicyclo[3.3.0]octane derivative (Patent Document 1), tea catechin (Patent Document 2), catechin and cacao polyphenol (Patent Document 3), asparagus extract (Patent Document 4) or the like as active ingredients, have been proposed.

Phytosterols are known to inhibit absorption of cholesterol in a gastrointestinal tract and exhibit an action of decreasing a blood cholesterol level. A cholesterol forms a micelle together with a bile acid and is solubilized, and then taken up by small intestinal epithelial cells. Phytosterols also form a micelle together with a bile acid. Since dissolution of the sterols in micelles is limited, coexisting phytosterols competitively inhibit the micelle formation of the cholesterol (Non-Patent Documents 1 and 2) It has been proposed to use phytosterols as body cholesterol-reducing agents using this action mechanism. For example, Patent Document 5 discloses a lipid metabolism improver comprising diglycerides or diglycerides and phytosterols as active ingredients in a cholesterol synthesis inhibitor-resistant hyperlipidemia patient. Patent Document 6 discloses a body cholesterol-reducing agent comprising an oil obtained from a soybean raw material with an embryo content of 15 wt % or more, as an active ingredient.

### PRIOR ART DOCUMENTS

### Patent Documents

Patent Document 1: Japanese Patent Application Laid-Open No. 2000-309533
Patent Document 2: Japanese Patent Application Laid-Open No. 2005-095186
Patent Document 3: Japanese Patent Application Laid-Open No. 2012-171916
Patent Document 4: Japanese Patent Application Laid-Open No. 2007-55951
Patent Document 5: Japanese Patent Application Laid-Open No. 2015-15425
Patent Document 6: WO 01/032032

### Non-Patent Documents

Non-Patent Document 1: Japan Food Research Laboratories, “About phytosterol”, Vol. 2, No. 57, November 2006
Non-Patent Document 2: Ishizaki et. al, “Comparison of cholesterol increase-inhibiting effects between soybean embryo-derived sterol and soybean sterol in rat”, Journal of Japan Society of Nutrition and Food Science, Vol. 58, No. 1, p. 11-16, 2005

### SUMMARY OF INVENTION

### Problem to be Solved

The object of the present invention is to provide a metabolic syndrome inhibitor which can inhibit metabolic syndrome by oral intake similarly to the prior art described above.

### Solution to Problem

The present inventors have found that a soybean hypocotyl oil has an action of inhibiting accumulation of body fats, particularly visceral fats and an action of suppressing increase of the blood neutral fat and thus it can be used for inhibiting metabolic syndrome, and this finding has led to the completion of the invention. That is, the present invention provides a metabolic syndrome inhibitor comprising a soybean hypocotyl oil as an active ingredient. The present invention is similar to the invention of Patent Document 6 in that they comprise the soybean hypocotyl oil as an active ingredient. However, the invention of Patent Document 6 is applied for the purpose of decrease of a body cholesterol level. The above-described action mechanism for decreasing the body cholesterol does not necessarily lead to inhibition of the body fat and visceral fat accumulation and inhibition of the blood neutral fat increase. Thus, Patent Document 6 disclosing only the body cholesterol-decreasing action does not teach a metabolic syndrome inhibitor which is an application of the present invention.

The metabolic syndrome inhibitor is particularly a body fat accumulation inhibitor and/or a blood neutral fat increase inhibitor.

The body fat accumulation inhibitor is particularly a visceral fat accumulation inhibitor.

The metabolic syndrome inhibitor preferably contains 1 to 100 wt % of the soybean hypocotyl oil.

Also, the present invention provides a fat or oil composition for inhibiting metabolic syndrome, which contains the metabolic syndrome inhibitor.

Also, the present invention provides a food for inhibiting metabolic syndrome, which contains the metabolic syndrome inhibitor, or is cooked using the inhibitor.

Also, the present invention provides a method for producing the metabolic syndrome inhibitor, including adding the soybean hypocotyl oil as an active ingredient.

Also, the present invention provides a method for producing the food for inhibiting metabolic syndrome, including cooking the food by adding the metabolic syndrome inhibitor comprising the soybean hypocotyl oil as an active ingredient to a foodstuff.

### Effects of Invention

The metabolic syndrome inhibitor of the present invention comprising soybean hypocotyl oil as an active ingredient exerts effects as a body fat accumulation inhibitor (in particular, a visceral fat accumulation inhibitor) and/or a blood neutral fat increase inhibitor, and as a result, metabolic syndrome can be improved or prevented. The soybean hypocotyl oil is known to have a blood cholesterol-decreasing action. The metabolic syndrome inhibitor of the present invention is superior to conventional metabolic syndrome inhibitors in that it simultaneously improves or prevents visceral fat type obesity and dyslipidemia associated with blood cholesterol and/or neutral fat. The metabolic syndrome inhibitor of the present invention can be greatly expected to prevent diseases developed due to progressed metabolic syndrome, e.g. arteriosclerosis, diabetic retinopathy, diabetic gangrene, diabetic nephropathy, renal failure, nephrosclerosis, uremia, angina pectoris, myocardial infarction, stroke, cerebral infarction and the like.

By the metabolic syndrome inhibitor of the present invention, dietary restriction is not required for humans. In particular, fats and oils are foodstuffs which stimulate human gustatory senses, and thus, if fats and oils in foods are restricted for reducing the lipid energy, human dietary life becomes insipid. On the other hand, since the metabolic syndrome inhibitor of the present invention comprises fats and oils as active ingredients, it can inhibit metabolic syndrome while maintaining affluent human dietary life.

### BRIEF DESCRIPTION OF DRAWINGS

FIG. 1 illustrates a diagram showing a feed intake of a food (feed) blended with the soybean hypocotyl oil mixture according to the present invention for mice compared to that of the soybean oil-blended feed. The intake of the feed during the test period in the soybean hypocotyl oil-mixed oil-blended feed group in Example 1 was larger than that in the soybean oil-blended feed group in Comparative Example 1.

FIG. 2 illustrates a diagram showing the body weight gain-inhibiting effects i.e. the body fat accumulation-inhibiting effects in the two groups in FIG. 1. The body weight gain was significantly inhibited by intake of the soybean hypocotyl oil-mixed oil-blended feed in Example 1.

FIG. 3 illustrates a diagram of the results of determining the feed efficiency (=weight gain/feed intake) from FIGS. 1 and 2. The feed efficiency in the soybean hypocotyl oil-mixed oil-blended teed group in Example 1 was lower. Since the feed intake in the test oil group is larger, it can be said that the test oil hardly increases the body weight even if fed in the same amount as in the soybean oil-blended feed group.

FIG. 4 illustrates a diagram showing the mesenteric fat weights during the dissection in the two groups in FIG. 1. The mesenteric fat in the soybean hypocotyl oil-mixed oil-blended feed group in Example 1 is decreased to about 50% of that in the soybean oil-blended teed group in Comparative Example 1.

FIG. 5 illustrates a diagram showing the epididymis fat weights during the dissection in the two groups in FIG. 1. The epididymis fat weight in the soybean hypocotyl oil-mixed oil-blended feed group in Example 1 is decreased to about 37% of that in the soybean oil-blended feed group in Comparative Example 1.

FIG. 6 illustrates a diagram showing the perirenal fat weights during the dissection in the two groups in FIG. 1. The perirenal fat weight in the soybean hypocotyl oil-mixed oil-blended feed group in Example 1 is decreased to about 60% of that in the soybean oil-blended feed group in Comparative Example 1.

FIG. 7 illustrates a diagram showing the posterior abdominal wall fat weights during the dissection in the two groups in FIG. 1. The posterior abdominal wall fat weight in the soybean hypocotyl oil-mixed oil-blended feed group in Example 1 is decreased to about 34% of that in the soybean oil-blended feed group in Comparative Example 1.

FIG. 8 illustrates a diagram of the results of determining the visceral fat weight (sum of weights of the above-described 4 fat sites) from FIGS. 4 to 7. The visceral fat weight in the soybean hypocotyl oil-mixed oil-blended feed group in Example 1 is decreased to about 39% of that in the soybean oil-blended feed group in Comparative Example 1.

FIG. 9 illustrates a diagram of the results of determining the visceral fat rate (=visceral fat weight/body weight) from FIGS. 2 and 8. It was revealed that the visceral fat rate in the soybean hypocotyl oil-Mixed oil-blended feed group in Example 1 was significantly decreased compared to that in the soybean oil-blended feed group.

FIG. 10 illustrates a diagram showing the liver weight during the dissection in the two groups in FIG. 1. There was no significant difference in the liver weight between the soybean hypocotyl oil-mixed oil-blended feed group in Example 1 and the soybean oil-blended feed group in Comparative Example 1.

FIG. 11 illustrates a diagram of the results of determining the liver rate (=liver weight/body weight) from FIGS. 2 and 10. There was no significant difference in the liver rate between the soybean hypocotyl oil-mixed oil-blended feed group in Example 1 and the soybean oil-blended feed group in Comparative Example 1.

FIG. 12 illustrates a diagram showing the thigh muscle weights during the dissection in the two groups in FIG. 1. The thigh muscle weight in the soybean hypocotyl oil-mixed oil-blended teed group in Example 1 was significantly higher than that in the soybean oil-blended feed group in Comparative Example 1.

FIG. 13 illustrates a diagram of the results of determining the muscle rate (=thigh muscle weight/body weight) from FIGS. 2 and 12. The muscle rate in the soybean hypocotyl oil-mixed oil-blended feed group in Example 1 was significantly higher than that in the soybean oil-blended feed group in Comparative Example 1.

FIG. 14 shows the measured values of the blood neutral fat (TG) during the rearing period in the two groups in FIG. 1. The TG in the soybean hypocotyl oil-mixed oil-blended feed group in Example 1 (-×-) transitioned low compared to the soybean oil-blended feed group in Comparative Example 1 (-▴-).

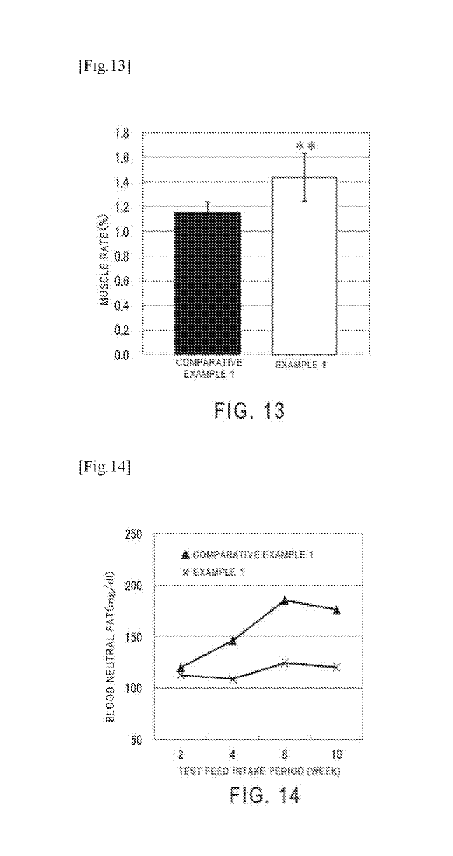

FIG. 13 illustrates a diagram showing the results of the relative values of the area values of the fat droplets in the unsaponifiable matter group and the avenasterol group relative to the area values of the fat droplets in a control group (DMSO), in the cell test. In the unsaponifiable matter group and the avenasterol group, fat accumulation in the fat cell was significantly inhibited compared to the control group.

### DESCRIPTION OF EMBODIMENTS

The metabolic syndrome inhibitor of the present invention (hereinafter referred to as the inhibitor of the present invention) comprises a soybean hypocotyl oil as an active ingredient. A soybean seed (whole soybean) is composed of seed leaf (about 90 wt %), seed coat (about 8 wt %) and hypocotyl (about 2 wt %). The soybean hypocotyl oil is an oil which is extracted and refined from a raw material having a higher ratio of hypocotyl in a soybean seed. The raw material obtained by increasing the ratio of the hypocotyl normally contains 15 to 80 wt % of hypocotyl, The oil extracted and refined from the raw material obtained in such a way normally contains 1500 to 6150 mg/100 g of unsaponifiable matter, 100 to 4000 mg/100 g of total Δ7 sterol, and 50 to 400 mg/100 g of avenasterol.

A method in which hypocotyl is selected from a whole soybean as a raw material and a soybean hypocotyl oil is extracted and refined therefrom is based on a conventional method. An example thereof will be outlined below. First, a soybean seed is heated to e.g. 40 to 80° C. Next, seed leaf, seed coat and hypocotyl are separated by removal, chop, crush or pulverization of the dried matter using a general-purpose pulverizing apparatus having at least one function of impact action, shearing action, compression/pressing action and frictional action. For the impact means, an impact mill, a jaw crusher, a stamp mill, a jet mill, a hammer mill, a pin mill, a tumbling mill, a satellite mill or the like is used; for the shearing means, a cutter mill, a stone mill or the like is used; for the compression/pressing means, a roller mill, a roll crusher, a press roll, a ring mill or the like is used; and for the friction means, a stream mill or the like is used.

Next, the mixture of the separated seed, seed leaf and hypocotyl is subjected to a separation means such as a vibration sieve, a rotary sieve and an air classifier, so that the seed coat and seed leaf are removed from the mixture to concentrate the hypocotyl. For example, a fraction which passed through a 7-mesh sieve is further fractionated to obtain a fraction between a 10-mesh sieve and a 14-mesh sieve. The fraction obtained in such a way normally contains 15 to 80 wt % of hypocotyl.

The fraction containing the hypocotyl obtained in the above step is heated at e.g. 40 to 100° C. for several seconds to about 60 minutes, then pressed into a flake, and this flake is brought into contact with an organic solvent such as n-hexane to obtain a crude raw oil. Furthermore, the crude raw oil is subjected to one or more steps of degumming, deoxidation, decolorization and deodorization, preferably steps of degumming, deoxidation, decolorization and deodorization to obtain soybean hypocotyl oil, in accordance with a conventional method. The soybean hypocotyl oil may be a commercial product.

An example of the compositions of the soybean hypocotyl oil and the soybean oil is shown in Table 1.

TABLE 1

| | | | | Soybean | |
|---|---|---|---|---|---|
| | | Soybean oil | | hypocotyl oil | |
| | Composition | (100 g) | | (100 g) | |
| Fatty acid | C16:0 (Palmitic acid) | 9.92 | g | 11.7 | g |
| | C18:0 (Stearic acid) | 4.23 | g | 3.38 | g |
| | C18:1 (Oleic acid) | 25.4 | g | 15.2 | g |
| | C18:2 (Linoleic acid) | 52.1 | g | 55.8 | g |
| | C18:3 (Linolenic acid) | 7.33 | g | 12.0 | g |
| | C20:0 (Arachidic acid) | 0.44 | g | 0.31 | g |
| | C20:1 (Eicosenoic acid) | 0.25 | g | 0.16 | g |
| | C22:0 (Behenic acid) | 0.35 | g | 0.27 | g |
| Tocopherol | α | 10.4 | mg | 61.4 | mg |
| | β | 1.52 | mg | 4.68 | mg |
| | γ | 64.9 | mg | 125.0 | mg |
| | δ | 17.7 | mg | 17.6 | mg |
| | Total tocopherol | 94.5 | mg | 208.7 | mg |
| Sterol | Campesterol | 62.1 | mg | 193 | mg |
| | Stigmasterol | 61.2 | mg | 179 | mg |
| | β-sitosterol | 184.4 | mg | 1968 | mg |
| | Δ7-stigmastenol | 28.7 | mg | 588 | mg |
| | Avenasterol | 10.1 | mg | 204 | mg |
| | Citrostadienol | 14.0 | mg | 639 | mg |
| | Total sterols (6 components) | 360.5 | mg | 3771 | mg |
| | Total Δ7 sterols | 52.8 | mg | 1431 | mg |

In this specification, the “total sterols” means the sum of 6 sterols consisting of β-sitosterol, campesterol, stigmasterol, Δ7-stigmastenol, avenasterol and citrostadienol. On the other hand, the “total Δ7 sterols” means the sum of 3 sterols consisting of Δ7-stigmastenol, avenasterol and citrostadienol. As shown in the above table, the soybean hypocotyl oil is characterized in that it has a higher rate of linoleic acid and linolenic acid, a higher content of the total sterols, and even a higher rate of the total Δ7 sterols compared to those of the soybean oil.

From the results of the cell test described below, it is preferable that the metabolic syndrome inhibitor comprising the soybean hypocotyl oil as an active ingredient contains 100 to 4000 mg/100 g of total Δ7 sterols, particularly 50 to 400 mg/100 g of avenasterol in order to exhibit a remarkable effect as a body fat accumulation inhibitor and a visceral fat accumulation inhibitor.

A base oil for diluting the soybean hypocotyl oil may be added to the inhibitor of the present invention as long as it does not inhibit the effect of the present invention. The base oil is not particularly limited as long as it is an edible fat or oil. Examples of the base oil include vegetable fats and oils such as palm kernel oil, palm oil, coconut oil, corn oil, cottonseed oil, soybean oil, rapeseed oil, rice oil, sunflower oil, safflower oil, cocoa butter and hypocotyl oil other than soybean hypocotyl oil (e.g. wheat hypocotyl oil, rice hypocotyl oil, and rapeseed hypocotyl oil), and animal fats and oils such as lard and fish oil, and the like. In addition, processed fats and oils thereof such as a fractionated oil (a medium melting point part of palm oil, a fractionated soft oil of palm oil, a fractionated hard oil of palm oil, etc.), a transesterified oil and a hydrogenated fat or oil can be used. In addition, each of these edible oils and fats may be used alone or in combination of two or more kinds.

Various additives can be blended into the inhibitor of the present invention. Examples of the additives include an emulsifier such as lecithin, monoglycerol fatty acid ester, organic acid monoglyceride, sorbitan fatty acid ester, propyleneglycol fatty acid ester, sucrose fatty acid ester, polyglycerol fatty acid ester and polysorbate; a flavor such as milk flavor, butter flavor, cheese flavor, yogurt flavor, coffee flavor, black tea flavor, cinnamon flavor and chamomile flavor; a flavor oil such as spearmint oil, clove oil and peppermint oil; a flavoring agent comprising an aldehyde such as acetaldehyde, benzaldehyde, butyraldehyde, citral, neral, decanal, ethylvanillin, vanillin, butyraldehyde and hexanal; a spice; an antioxidant such as tocopherol, L-ascorbic acid (e.g. L-ascorbyl palmitate), butylhydroxyanisole (BHA), butylhydroxytoluene (BHT), tertiary butylhydroquinone (TBHQ), catechin, lignan and γ-oryzanol; an antifoaming agent such as silicone; a physiologically active substance such as a fatty acid such as DHA and EPA, vitamin A, vitamin D and coenzyme Q, and the like.

The content of soybean hypocotyl oil in the inhibitor of the present invention is normally 1 to 100 wt %, preferably 3 to 100 wt %, particularly preferably 5 to 100 wt %.

The oil content (sum of soybean hypocotyl oil and base oil) in the inhibitor of the present invention is normally 1 to 100 wt %, preferably 3 to 100 wt %.

The form of the inhibitor of the present invention may be liquid, emulsion (water-in-oil (W/O) type or oil-in-water (O/W) type), solid (powder, granule, flake, block, etc.). The inhibitor of the present invention is preferably composed of a liquid or an emulsion.

The inhibitor of the present invention can be prepared in accordance with an appropriate method depending on the form. For example, a liquid or solid inhibitor can be obtained by mixing the soybean hypocotyl oil with an appropriate base oil and additives. The inhibitor can be adjusted so as to be liquid or solid depending on selection of the base oil.

An inhibitor in an emulsion form can be obtained by stirring and mixing a mixture containing e.g. a soybean hypocotyl oil, an edible fat or oil (base oil), an emulsifier, other additives and water by using an emulsifier or the like. The oil content in the emulsion is normally 10 to 90 wt %.

The powdery or granular inhibitor can be obtained by a process that an emulsion obtained by stirring and mixing a soybean hypocotyl oil with a mixture containing an edible fat or oil (base oil), an emulsifier, a powdered base and water is further dried and powdered, for example. The drying and powdering includes emulsion spray drying and the like, for example.

The present invention particularly provides a fat or oil composition for inhibiting metabolic syndrome, comprising the metabolic syndrome inhibitor of the present invention. The base oil of the fat or oil composition is not particularly limited as long as it is a fat or oil used for foods. Such a fat or oil is the same as the above examples for the base oil of the inhibitor. The base oil of the oil and fat composition may be the same as or different from the base oil of the inhibitor. Preferably, the base oil is soybean oil, rapeseed oil, corn oil, palm oil, rice oil, olive oil and sesame oil.

The fat or oil composition of the present invention can be blended with the above examples for the additives of the general-purpose inhibitor as the edible fats and oils.

The content of the inhibitor in the fat or oil composition of the present invention is normally 1 to 100 wt %, preferably 3 to 100 wt %, particularly preferably 5 to 100 wt % as the soybean hypocotyl oil.

The oil content (sum of soybean hypocotyl oil and base oil) in the fat or oil composition of the present invention is usually 50 to 100 wt %, preferably 60 to 100 wt %, more preferably 80 to 100 wt %, still more preferably 90 to 100 wt %.

Also, the present invention provides foods (including feeds) for inhibiting metabolic syndrome. The foods for inhibiting metabolic syndrome include foods containing the inhibitor (hereinafter referred to as processed foods) or foods cooked using the inhibitor (hereinafter referred to as cooked foods).

Specific examples of the processed foods and cooked foods include tempura, deep-fried chicken, okonomiyaki, Korean pancake, pancake, donut, modified milk powder, roux (curry, stew, hash, etc.), instant cooked foods and drinks (instant noodle, instant soup, instant miso soup, instant coffee, instant cocoa, etc.), retort foods (curry, stew, pasta sauce, soup etc.), chilled and frozen foods (donut, French fries, deep-fried chicken, croquette, minced meat fried cake, pork cutlet, fried fish, fried calamari rings, fried onion rings, gratin, pizza, fried rice, pilaf, Japanese wheat noodle, ramen noodle, meat bun, jaozi, etc.), processed meat products (ham, bacon, sausage, hamburg steak, roast pork, seasoned meat, roast beef, steak etc.), processed seafood products (fish sausage, boiled fish-paste, spicy cod roe, minced tuna welsh onion, salted fermented seafood, shrimp paste, etc.), seasonings (miso, Worcestershire sauce, tomato sauce, seasoning sauce, mayonnaise, salad dressing, ponzu vinegar, flavor oil, Chinese cooking mix, source of chicken stock, bouillon, pot cooking soup, etc.), confectionery/bakery (potato chips, chocolate, cookie, cake, pie, biscuit, cracker, gummi candy, chewing gum, nougat, toffee, caramel, candy, tablet confectionery, bread, danish pastry, etc.), confectionery materials (chocolate spread, chocolate coating, and dessert mix such as almond jelly mix, pudding mix and jelly mix, etc.), supplements (tablet, capsule, solid, liquid, powder, etc.), health foods (soft drink, green leafy vegetable juice, cereal, cereal bar, etc.), dairy products (milk beverage, fermented milk beverage, butter, cream, processed cheese, cheese processed food, etc.), dairy product substitutes (margarine, shortening, fat spread, non-dairy creamer, coffee cream, whipped cream, etc.), cold confectioneries (ice cream, jelly, pudding, etc.) and the like.

The above-mentioned processed foods can be produced in accordance with a conventional method depending on the foodstuff to be used and its form, except that the inhibitor of the present invention is added.

The inhibitor of the present invention is added to e.g. an inside or a surface of a foodstuff, a batter liquid, a blender liquid, a pickling liquid, the tumbling liquid, and the like.)

The amount of the inhibitor of the present invention added to the processed food is normally 1 to 90 wt % preferably 1 to 85 wt %, more preferably 1 to 80 wt %, particularly preferably 1 to 75 wt % as a content of the soybean hypocotyl oil.

The above-described cooked foods can be cooked or produced in accordance with a conventional method regardless of the materials to be used and without special conditions, except that the food is cooked or produced using the inhibitor of the present invention.

Specific examples of the cooking methods for the foods include: a method of deep-frying tempura, potato, chicken, croquette, minced meat cake, pork cutlet, fish, calamari rings, onion rings or the like; a method of stir-frying beef, pork, chicken, rice, pilaf, vegetable, fish, noodles or the like; a method of boiling meat, vegetable, fish, beans or the like; a method of pouring the food to meat, vegetable, fish, beans, pizza, ramen noodle, Japanese wheat noodle or the like; a method of spreading the food to bread, confectionery or the like; a method of putting the food to bread, confectionery or the like; and the like.

The above-described metabolic syndrome inhibitor of the present invention, and the oil and fat composition and the food containing the inhibitor, or the food cooked using the inhibitor are effective for inhibiting metabolic syndrome, particularly body fat accumulation, visceral fat accumulation, and/or blood neutral fat increase.

### EXAMPLES

Hereinafter. Examples and Comparative Examples of the present invention will be described, but the present invention is not limited thereto.

### (Test Samples)

In this example, a soybean hypocotyl oil and a soybean hypocotyl oil-mixed oil were prepared and used as test samples. The preparation method is as follows.

### (Method for Preparing Soybean Hypocotyl Oil)

Soybean seeds were heated at 80° C. for 45 minutes and pulverized to less than half of the original size to obtain a Mixture of seed leaf, seed coat and hypocotyl. The resulting mixture was subjected to an air classifier to remove seed coat, to obtain a mixture of seed leaf and hypocotyl. The resulting mixture of seed leaf and hypocotyl was subjected to sieving machine to remove a fraction which had not passed through a 7-mesh sieve, and furthermore a fraction between a 10-mesh sieve and a 14-mesh sieve was isolated to obtain a hypocotyl fraction (soybean hypocotyl: 40 wt %).

The hypocotyl fraction was heated to 60° C., flaked by a press machine, and an oil content was extracted with n-hexane to obtain a micella. From the resulting micella, the residual n-hexane was removed under reduced pressure at 60 to 80° C. to obtain a crude raw oil. To the crude raw oil, 0.1% of phosphoric acid was added, then stirred at 70° C. for 15 minutes, to which distilled water was added, stirred at 70° C. for 30 minutes, and then centrifuged to remove a gum content (degumming). Next, 0.1% of phosphoric acid was added, then the mixture was stirred at 75° C. for 15 minutes, to which a sodium hydroxide aqueous solution was added, stirred for 20 minutes, and then centrifuged. Next, distilled water was added in an amount equivalent to 5%, the mixture was washed at 80° C. for 1 minute, and centrifuged (deoxidation). Next, 2% of activated clay was added, the mixture was stirred under reduced pressure at 80° C. for 30 minutes, and then filtered (decolorization). Subsequently, the mixture was distilled with steam (steam amount: 2%) at 180° C. for 30 minutes (deodorization) to obtain a soybean hypocotyl oil.

### (Method for Preparing Soybean Hypocotyl Oil Mixture)

50 parts by weight of the soybean hypocotyl oil was mixed with 50 parts by weight of soybean oil (product name: Soybean refined oil NS, made by J-OIL MILLS, Inc.) to obtain a soybean hypocotyl oil mixture.

### Example 1

### Evaluation of Metabolic Syndrome Inhibitor Efficacy by Animal Test and Cell Test

The body fat accumulation-inhibiting action, the visceral fat-inhibiting action and the blood neutral fat increase-inhibiting action of the soybean hypocotyl oil as an active ingredient of the metabolic syndrome inhibitor of the present invention were investigated in an animal test. Furthermore, components of the soybean hypocotyl oil which affect the body fat accumulation-inhibiting action and the visceral fat-inhibiting action were investigated by a cell test.

### A. Animal Test

### (1) Preparation of Feed

As the metabolic syndrome inhibitor to be blended into the feed, the soybean hypocotyl oil mixture was used. Also, the soybean oil (unsaponifiable matter content: 430 mg/100 g) was prepared for comparison. Phytosterols in the above-described two oils were analyzed. The results of the phytosterol concentrations and sterol compositions in the oils are shown in Table 2.

TABLE 2

| | | | Soybean hypocotyl | |
|---|---|---|---|---|
| | Soybean oil | | oil-mixed oil | |
| | | Sterol | | Sterol |
| | | composition | | composition |
| | mg/100 g | (wt %) | mg/100 g | (wt %) |
| Campesterol | 70 | 20.2 | 169 | 8.4 |
| Stigmasterol | 61 | 17.5 | 112 | 5.5 |
| β-sitosterol | 182 | 52.2 | 1130 | 55.8 |
| Δ7-stigmastenol | 21 | 6.1 | 769 | 13.3 |
| Avenasterol | 3 | 0.8 | 68 | 3.4 |
| Citrostadienol | 11 | 3.2 | 275 | 13.6 |
| Total sterols | 348 | 100 | 2023 | 100 |
| (6 components) | | | | |
| Total Δ7 sterols | 35 | 10.1 | 612 | 30.3 |

In accordance with the composition shown in Table 3, the compositions were mixed by a kitchen-aid mixer (KSM5, made by FMI Corporation) for 15 minutes to prepare a feed for preliminary rearing, a soybean oil-blended feed (Comparative Example 1), and a soybean hypocotyl oil-mixed oil-blended feed (Example 1).

TABLE 3

| | | | Example 1 |
|---|---|---|---|
| | | Comparative | (Soybean |
| | Feed for | Example 1 | hypocotyl |
| | preliminary | (Soybean oil- | oil-mixed oil- |
| Composition | rearing | blended feed) | blended feed) |
| Soybean oil | 5 | 20 | — |
| Soybean hypocotyl oil- | — | — | 20 |
| mixed oil | | | |
| Casein | 20 | 20 | 20 |
| Corn starch | 30 | 15 | 15 |
| Sucrose | 35 | 35 | 35 |
| Cellulose | 5 | 5 | 5 |
| AIN-93 mineral MIX | 4 | 4 | 4 |
| AIN-93 vitamin MIX | 1 | 1 | 1 |
| Methionine | 0.3 | 0.3 | 0.3 |
| Choline bitartrate | 0.2 | 0.2 | 0.2 |
| Total | 100 | 100 | 100 |

Casein: Casein, derived from milk, made by Wako Pure Chemical Corporation
Corn starch: Corn starch Y, made by J-OIL MILLS, Inc.
Sucrose: Granulated sugar, made by Mitsui Sugar Co., Ltd.
Cellulose: VIVAPUR Type 102, made by JRS PHARMA, Inc.
AIN-93 mineral MIX: AIN-93M-MX, made by CLEA Japan, Inc.
AIN-93 vitamin MIX: AIN-93VX, made by CLEA Japan, Inc.
Methionine: DL-methionine, made by Wako Pure Chemical Corporation
Choline Bitartrate: made by NACALAI TESQUE, INC.

### (2) Animal Administration Test

Male C57BL/6j mice aged 7 weeks were purchased from Charles River Laboratories Japan, Inc., and preliminarily reared using the feed for preliminary rearing for 6 days. After the preliminary rearing, mice were divided into groups including 6 mice per one group so that there was no difference in the average body weight for each group. After the grouping, the mice were fed with the test feed comprising the soybean hypocotyl oil-mixed oil-blended feed or the soybean oil-blended teed for 12 weeks. During the preliminary rearing period and the test feed intake rearing period, mice were reared under an environment of a temperature of 23° C±2° C., a humidity of 40 to 60%, a light period of 7:30 to 19:30, and a dark period of 19:30 to 7:30. In addition, the feed and water were freely ingested. The body weight was measured once a week during the rearing period. Also, during the rearing period, the blood neutral fat was measured every 2 weeks.

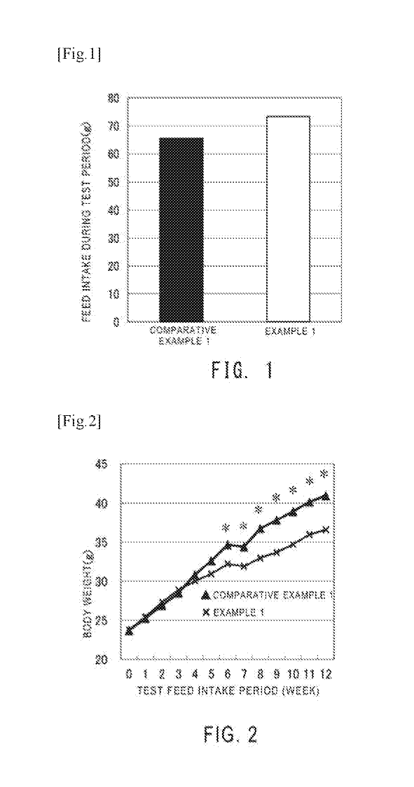

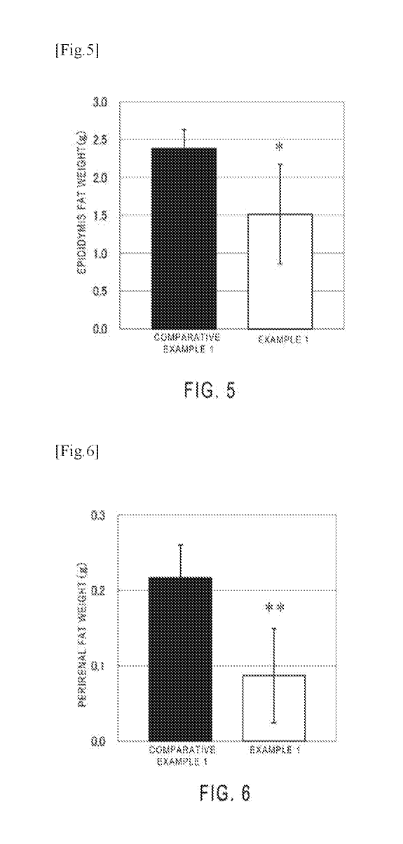

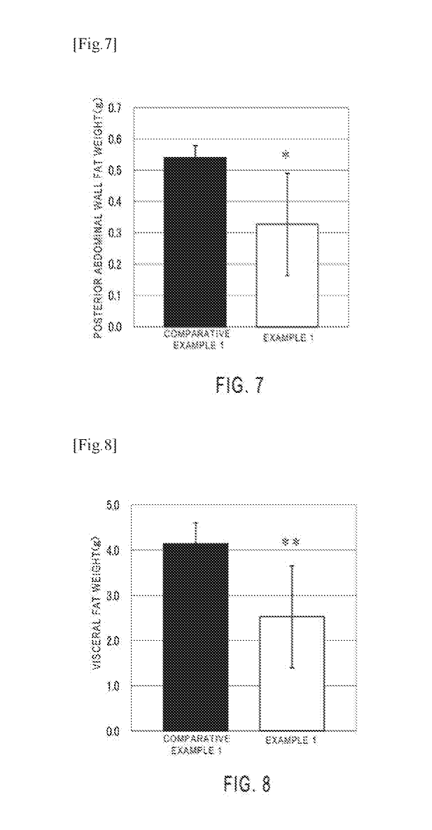

Fasting was started at 17 o'clock on the day before the last day of the test feed intake. On the last day, laparotomy was performed under deep anesthesia with sodium pentobarbital, epididymis fat, mesenteric fat, perirenal fat, posterior abdominal wall fat, liver and thigh muscle were removed, and their weights were measured. A body weight, a blood neutral fat value, weights of epididymis fat, mesenteric fat, perirenal fat, posterior abdominal wall fat and visceral fat (sum of the above-described 4 fats), visceral fat rate (=visceral fat weight/body weight), a liver weight and a liver rate (=liver weight/body weight), a thigh muscle weight and a muscle rate (=thigh muscle weight/body weight) were statistically processed (unpaired t-test). Among FIGS. 1 to 13, examples for which a significant difference had been confirmed were represented * or **. Note that * indicates a risk rate (p value) of less than 0.05, and ** indicates a risk rate (p value) of less than 0.01.

FIG. 1 shows the results of comparing feed intakes during the test. The black bar graph indicates the soybean oil-blended feed group, and the white bar graph indicates the soybean hypocotyl oil-mixed oil-blended feed group. As shown in FIG. 1, the intake of the feed during the test period in the soybean hypocotyl oil-mixed oil-blended feed group in Example 1 was larger than that in the soybean oil-blended feed group in Comparative Example 1.

FIG. 2 shows weight gain-inhibiting effects in the soybean hypocotyl oil-mixed oil-blended feed group (-×-) and the soybean oil-blended feed group (-▴-). As shown in FIG. 2, the body weight gain was significantly inhibited in the soybean hypocotyl oil-mixed oil-blended feed group.

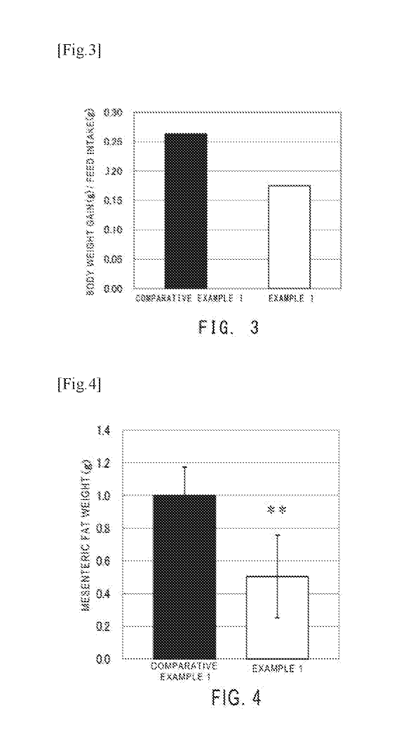

FIG. 3 shows the results of determining the feed efficiency body weight gain/feed intake) from FIGS. 1 and 2. As shown in FIG. 3, the feed efficiency in the soybean hypocotyl oil-mixed oil-blended feed group was lower than that in the soybean oil-blended feed group. Since the feed intake in the soybean hypocotyl oil-mixed oil-blended feed group is larger, it can be said that the soybean hypocotyl oil-mixed oil-blended feed hardly increases the body weight even if fed in the same amount as in the soybean oil-blended feed group.

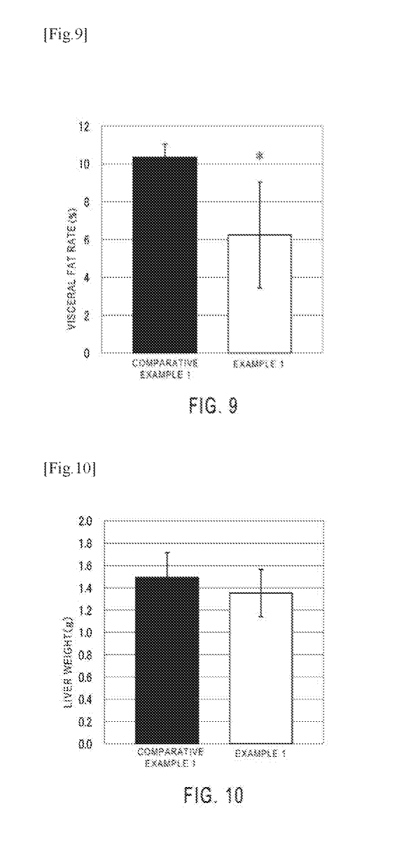

FIGS. 4 to 7 show various fat weights during dissection, and FIG. 8 shows the sum of visceral fat weights of the above-described 4 fat sites during dissection. FIG. 9 shows the visceral fat rate represented by a visceral fat weight/body weight during dissection. As shown in FIGS. 4 to 9, it was found that the visceral fat weight and the visceral fat rate in the soybean hypocotyl oil-mixed oil-blended feed group were significantly decreased compared to the soybean oil-blended feed group.

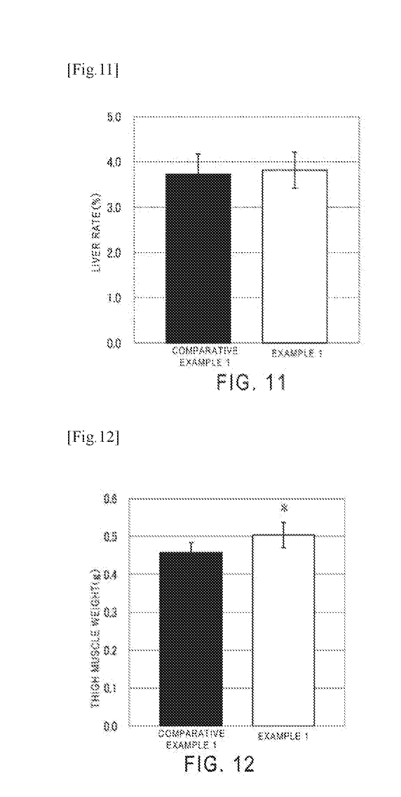

FIGS. 10 and 11 show the liver weights and the liver rates (=liver weight/body weight) during dissection in the soybean hypocotyl oil-mixed oil-blended feed group and the soybean oil-blended feed group respectively. There was no significant difference in the liver weight and the liver rate between the both groups.

FIGS. 12 and 13 show the thigh muscle weights and the muscle rates (=thigh muscle weight/body weight) during dissection in the soybean hypocotyl oil-mixed oil-blended feed group and the soybean oil-blended feed group respectively. The thigh muscle weight and the muscle rate in the soybean hypocotyl oil-mixed oil-blended feed group were significantly higher than those in the soybean oil-blended feed group.

In the soybean hypocotyl oil-mixed oil-blended feed group, despite a large feed intake, the body weight gain was inhibited more than in the soybean oil-blended feed group (FIGS. 1 to 3). In the soybean hypocotyl oil-mixed oil-blended feed group, the accumulation of body fat and visceral fat is significantly inhibited (FIGS. 4 to 9). Meanwhile, in the soybean hypocotyl oil-mixed oil-blended feed group, organs and muscles are not decreased (FIGS. 10 to 13). Consequently, it can be said that the body weight gain-inhibiting effect caused by the inhibitor of the present invention shown in FIG. 2 is based on the inhibition of the body fat and visceral fat accumulations.

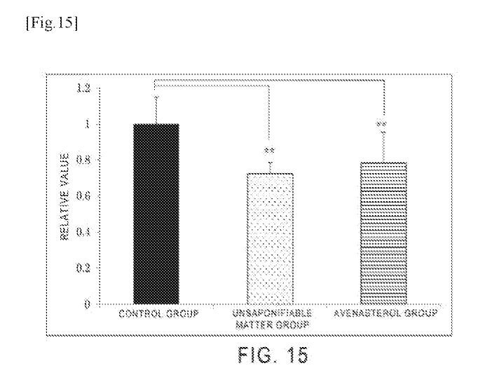

FIG. 14 shows the measured values of the blood neutral fat during the rearing period in the soybean hypocotyl oil-mixed oil-blended feed group (-Δ-) and the soybean oil-blended feed group (-▴-). The blood neutral fat value in the soybean hypocotyl oil-mixed oil-blended feed group (-Δ-) transitioned low compared to the soybean oil-blended feed group (-▴-). Conventionally, it has been revealed that the component of the soybean hypocotyl oil has a blood cholesterol-lowering action. In this test, it was also revealed that the soybean hypocotyl oil had a blood neutral fat increase-inhibiting action. Thus, it was revealed that the metabolic syndrome inhibitor of the present invention simultaneously improved or prevented visceral fat obesity and dyslipidemia associated with abnormalities of blood cholesterol and/or blood neutral fat.

### B. Cell Test

### (1) Preparation of Test Substance

A test was carried out about whether the phytosterol components in the soybean hypocotyl oil provided the visceral fat accumulation-inhibiting action. A soybean hypocotyl oil (unsaponifiable matter content: 4710 mg/100 g) was obtained by the same operation as for the soybean hypocotyl oil used in the animal test. The results of analyzing phytosterols in the soybean hypocotyl oil are shown in Table 4.

TABLE 4

Soybean hypocotyl oil

| | | Sterol composition |
|---|---|---|
| | mg/100 g | (wt %) |
| Campesterol | 193 | 5.1 |
| Stigmasterol | 179 | 4.8 |
| β-sitosterol | 1968 | 52.7 |
| Δ7-stigmastenol | 588 | 15.6 |
| Avenasterol | 204 | 5.4 |
| Citrostadienol | 639 | 16.9 |
| Total sterols (6 components) | 3771 | 100 |
| Total Δ7 sterols | 1431 | 37.9 |

Unsaponifiable matter was obtained from the soybean hypocotyl oil in accordance with the following procedure. First, 3 g of soybean hypocotyl oil was precisely weighed in a 300 mL Erlenmeyer flask with a stopper, to which 25 mL of a 2 mol/L potassium hydroxide/ethanol solution, and 25 mL of a 0.05 g/mL gallic acid/ethanol solution were added. Two boiling tips were added to the Erlenmeyer flask, to which a Soxhlet extractor cooling pipe with circulating cooling water was connected, and the flask was heated in a steam generated by a water bath for 1 hour to cause saponification reaction.

The saponification reaction solution was transferred to a 500 mL separatory funnel, the saponification reaction solution remaining in the Erlenmeyer flask was co-washed with 100 mL of hot water, and transferred to the separatory funnel. 50 mL of pure water at normal temperature was added, and the mixture was allowed to stand and cooled until it reached room temperature. While washing the Erlenmeyer flask with 100 mL of diethyl ether, the diethyl ether was put into the separatory funnel. The separatory funnel was capped, the mixture was shaken vigorously for 1 minute, and allowed to stand until the mixture was separated into two layers of water and diethyl ether. The lower layer (water layer) was removed, 30 mL of pure water was added to the funnel, which was capped, and gently rotated 2 to 3 times in such a way that the whole inner wall of the separatory funnel was washed by the water layer portion, allowed to stand to separate the mixture into two layers, and then the lower layer (water layer) was removed. 30 mL of pure water was added to the separatory funnel again, which was capped, gently rotated 2 to 3 times in such a way that the whole inner wall of the separatory funnel was washed by the water layer portion, allowed to stand to separate the mixture into two layers, and then the lower layer (water layer) was removed. 30 mL of pure water was added to the funnel, the mixture was thoroughly shaken, then allowed to stand until the mixture was separated into two layers, and the lower layer (water layer) was removed. This operation was repeated until the removed lower water layer solution was no longer colored with a phenolphthalein solution. When the lower layer was no longer colored, the upper layer (diethyl ether layer) was taken in a beaker, dehydrated with anhydrous sodium sulfate, then the diethyl ether solution was transferred to a 300 mL eggplant flask, and diethyl ether was removed by a rotary evaporator. Drying treatment was performed under a reduced pressure of 25 to 30 KPa at 60° C. for 30 minutes. After cooled in a desiccator, the resulting extract was specified to be unsaponifiable matter The above obtention procedure was repeated to obtain unsaponifiable matter for the cell test from the soybean hypocotyl oil.

The results of analyzing phytosterols in 14.65 mg of the above unsaponifiable matter are shown in Table 5.

TABLE 5

Soybean hypocotyl oil
unsaponifiable matter

| | mg | Composition (wt %) |
|---|---|---|
| Unsaponifiable matter | 14.65 | 100 |
| Campesterol | 0.82 | 5.6 |
| Stigmasterol | 0.63 | 4.3 |
| β-sitosterol | 7.01 | 47.9 |
| Δ7-stigmastenol | 1.26 | 8.6 |
| Avenasterol | 0.50 | 3.4 |
| Citrostadienol | 1.36 | 9.3 |
| Total sterol (6 components) | 11.58 | 79.1 |
| Total Δ7 sterols | 3.07 | 20.9 |

In accordance with the following procedure, an avenasterol fraction was obtained from the above unsaponifiable matter. First, about 40 mg of the unsaponifiable matter was dissolved in 4 mL of tetrahydrofuran, which was sonicated for 1 minute. 140 μL of the unsaponifiable matter solution was poured into a 5C18AR column (20 mm (I.D.)×250 mm, particle size: 5 μm, made by Waters Corporation), and flowed with a solution of methanol:acetonitrile:tetrahydrofuran=1:2:0.1 at a flow rate of 20 mL/min, and the avenasterol was separated while confirming an absorbance at 210 nm to obtain an avenasterol solution. The resulting avenasterol solution was passed through the column again to obtain a high-concentration avenasterol solution. From the resulting high-concentration avenasterol solution, the solvent was removed using a rotary evaporator. The solution was dried in a vacuum dryer at 60° C. for 30 minutes to obtain an avenasterol fraction. A purity of the avenasterol in the avenasterol fraction was 87.2 wt %. Citrostadienol was not detected from the avenasterol fraction.

### (2) Method for Preparing Test Solution

100 μL of dimethylsulfoxide (DMSO) was added to 4.147 mg of unsaponifiable matter, or 259.65 μL of dimethylsulfoxide (DMSO) was added to 1.0716 mg of the avenasterol fraction, and which was sonicated for 1 minute to obtain a test solution. The test solution of the unsaponifiable matter (avenasterol concentration: 1.41 mg/ml) was referred to as an unsaponifiable matter group, and the test solution of the avenasterol (avenasterol concentration: 3.60 mg/ml) was referred to as an avenasterol fraction group. Furthermore, a DMSO without the unsaponifiable matter nor the avenasterol fraction (control group) was prepared. Each test solution in each group was diluted 1000-fold with a medium described in the following “Cell test”, and sonicated for 1 minute to obtain a medium with the test solution.

### (3) Cell Test

The cell test was carried out using a visceral fat cell culture kit (VAC21, made by Cosmo Bio Company, Limited) in accordance with the attached protocol. Specifically, 3.0×10⁶ rat primary visceral fat cells dissolved in a hot water bath at 37° C. were seeded in a 24-well plate, and precultured in a medium attached to the kit for 4 days After the preculture, the medium was removed, the cells were cultured in 1 mL of a medium with the test solution for 2 days, and after 2 days, the medium was removed, the cells were cultured in 1 mL of the medium with the test solution for 2 more days.

### (4) Oil Red O Staining

The medium was removed, and the inside of the well was washed with 0.5 mL of phosphate buffered saline (PBS). 0.5 mL of 10% formalin solution was added to each well and the cells were fixed at room temperature for 10 minutes. Formalin was removed, and the inside of the well was washed with 0.5 mL of PBS. 0.5 mL of 60% isopropanol was added to each well, which was allowed to stand at room temperature for 1 minute. 60% isopropanol was removed. The well was allowed to stand with 0,5 mL of Oil Red O stain liquid (0.3 g Oil Red/100 mL isopropanol) at room temperature for 20 minutes. The Oil Red O stain liquid was removed, 0.5 mL of 60% isopropanol was added to each well, which was allowed to stand for 1 minute. The 60% isopropanol was removed, and the inside of the well was washed with 0.5 mL of PBS. The cells were microscopically observed at a magnification of 200 times, and visceral fat cells with stained fat droplets were recorded as images on a recording device of a computer.

### (5) Analysis of Fat Droplet Area Value

Area values of the lipid droplets stained with the Oil Red O on the above-described images were measured using the image processing software ImageJ (1.48 v) (obtained from https://imagej.nih.gov/ij/). The cell test was carried out three times, and four images were recorded in each group for each cell test. For each cell test, an average value of the area value of the fat droplets in each object group was calculated to calculate a relative value of the area value (hereinafter referred to as a relative value) of the fat droplets on each image when the average area value of the fat drops on the control group was taken as 1. 12 relative values for each group obtained in this manner was statistically processed (Tukey-Kramer test). FIG. 15 shows the resultant relative values in the unsaponifiable matter group and the avenasterol group relative to those in the control group. In addition, ** indicates the risk value (p value) of 0.01 or less.

As shown in FIG. 15, in the unsaponifiable matter group and the avenasterol group, fat accumulation in the fat cell was significantly inhibited compared to the control group. Thus, there is high possibility that one of the active ingredients in the unsaponifiable matter is avenasterol.

From the results of the cell test described above, it was confirmed that the metabolic syndrome inhibitor comprising the soybean hypocotyl oil as an active ingredient preferably contained the Δ7 sterol, particularly the avenasterol in order to exhibit effects of inhibiting the body fat accumulation and the visceral fat accumulation.

1-4. (canceled)

5. A fat or oil composition for inhibiting metabolic syndrome, containing a metabolic syndrome inhibitor comprising a soybean hypocotyl oil as an active ingredient, wherein the metabolic syndrome is body fat accumulation.

6. A food for inhibiting metabolic syndrome, containing a metabolic syndrome inhibitor comprising a soybean hypocotyl oil as an active ingredient, or is cooked using the inhibitor, wherein the metabolic syndrome is body fat accumulation.

7. A method for producing a metabolic syndrome inhibitor, including adding a soybean hypocotyl oil as an active ingredient, wherein the metabolic syndrome is body fat accumulation.

8. A method for producing a food for inhibiting metabolic syndrome, including cooking the food by adding a metabolic syndrome inhibitor comprising a soybean hypocotyl oil as an active ingredient to a foodstuff, wherein the metabolic syndrome is body fat accumulation.

9. A method for inhibiting metabolic syndrome, including ingesting a metabolic syndrome inhibitor comprising a soybean hypocotyl oil as an active ingredient, wherein the metabolic syndrome is body fat accumulation.

10. The method for inhibiting metabolic syndrome as claimed in claim 9, wherein the body fat accumulation is visceral fat accumulation.

11. The method for inhibiting metabolic syndrome as claimed in claim 9, wherein the metabolic syndrome inhibitor contains 1 to 100 wt % of the soybean hypocotyl oil.
